SEQUENCE LISTING (10 sequences)

SEQ ID NO: 1 - DNA, 366 bp, Echinacea purpurea
tcgaggtcatctcttcattaccacaaacgtactacaactcgttcgtcttgtcttccacaaacgattaaacatatagaaaaacttgatgttgtttcagactctcatctccgttagcacaactacggttgctttaccttggcagacatatacgcaacctaaagaaaaatataaaaatttattttaagagatccgtgttaaagagattcatgatcaacatctgacgtctcttacatgctcattcttgaaggtgtaaacggtattatgttttattgttatccaaacaaaacctctaaggataaggtttagtttgttttcataatggtcatggtcaataacgtgttagaaacgtgaaaagtgcatctcata

SEQ ID NO: 2 - DNA, 528 bp, Artificial Sequence
  Synthetically generated oligonucleotide
tcgactacttacaacctattgaatgatagttgtttctagcacaagaaattgttctctgacttgtaaaatttagacaacttttgttatacaaattagtcaaattgttaacaaagataagctacttaaagcatctaaaatgtacaaaaagttgtaatgtgtattagacaaacacatacaaaggataaaagtccatccataagaaatatgataaatgttaaaagacaatgtgaagttataaattcagtcacatgtatgtttctatctcccccttgtcaccgttattaatgttattctgggattcatcagtcatcccttgagatatttaatcctagcttaactcttaatgacgatgtgaaactataaattcagtcacatgtatgatgctgacatatgaaatttatatattgcttcgtggcaaatgttcattaaatgtgtaataaagatggactagatttggtcccaagttgagccagttttctacaatgtgtgaacagaccatgtctacttttttggcagccgttgctcacc

SEQ ID NO: 3 - DNA, 21 bp, Artificial Sequence
  Primer
ttgtcaagaccgacctgtccg

SEQ ID NO: 4 - DNA, 21 bp, Artificial Sequence
  Primer
cgttgcggttctgtcagttcc

SEQ ID NO: 5 - DNA, 18 bp, Artificial Sequence
  Primer
agattagccttttcaatt

SEQ ID NO: 6 - DNA, 18 bp, Artificial Sequence
  Primer
ttaagtagcaacactgtg

SEQ ID NO: 7 - DNA, 18 bp, Artificial Sequence
  Primer
atgattgaacaagatgga

SEQ ID NO: 8 - DNA, 18 bp, Artificial Sequence
  Primer
tcagaagaactcgtcaag

SEQ ID NO: 9 - DNA, 18 bp, Artificial Sequence
  Primer
atggtgacagtcgaggag

SEQ ID NO: 10 - DNA, 18 bp, Artificial Sequence
  Primer
ttaagtagcaacactgtg

(19) United States
(12) Patent Application Publication
(10) Pub. No.: US 2006/0075525 A1
(43) Pub. Date: Apr. 6, 2006

(54) Transgenic Echinacea plants

(76) Inventors: Kin-Ying To, Taipei (TW); Hsin-Mei Wang, Taipei (TW)

Correspondence Address: FISH & RICHARDSON PC, P.O. BOX 1022, MINNEAPOLIS, MN, 55440-1022, US

(21) Appl. No.: 10/958,983

(22) Filed: Oct. 5, 2004

(51) Int. Cl. A01H 1/00; C12N 15/82; C12N 5/04

(52) U.S. Cl. 800/294; 435/468; 435/419

(57) ABSTRACT

A method of producing a transformed Echinacea plant cell or a transgenic Echinacea plant. Also disclosed are a transformed Echinacea plant cell and a transgenic Echinacea plant containing a recombinant nucleic acid.

### BACKGROUND

Plants are sources of drugs and other useful substances. For example, Echinacea sp., a member of the Aster family (Compositae or Asteraceae), has been used as a medicinal plant for hundreds of years.

Among nine varieties of the Echinacea plant, most commonly used are Echinacea angustifolia DC var. angustifolia, E. pallida (Nutt.) Nutt., and E. purpurea (L.) Moench. See Nieri et al., Planta Med. 2003, 69:685-686. Extracts from roots and aerial parts of E. angustifolia and E. purpurea (commonly known as purple coneflower) have been used to treat wounds, snake bites, headaches, infections, and common colds, and to stimulate the immune system. See, e.g., Barrett, Phytomedicine, 2003, 10:66-86. Possible active ingredients include flavonoids, essential oils, polysaccharides, caffeic acid derivatives, alkylamides, and other compounds. Among them, flavonoids, a class of secondary metabolites in plants, possess antioxidantive and antimicrobial activities (Arts, et al., J. Agric. Food Chem. 2002, 50:1184-1187 and O'Byrne et al., Am. J. Clin. Nutr. 2002, 76:1367-1374). In addition, they protect plants from UV damages, play key roles in signaling between plants and microbes, and provide pigmentation in flowers, fruits, seeds, and leaves. See Forkmann et al., Curr. Opin. Biotechnol. 2001, 12:155-160 and B. Winkel-Shirley, Plant Physiol. 2001, 126:485-493. Further studies are needed for elucidating activities and action mechanisms of flavonoids and other useful substances. Nonetheless, they are hindered by availability of these useful substances.

Genetic engineering has been used as a relatively fast, precise, and cost-effective means of achieving desired characteristics in certain plants. However, genetic transformation of Echinacea sp has not been successful.

### SUMMARY

This invention relates to introducing into an Echinacea plant or cell a heterologous nucleic acid, e.g., the gene encoding a chalcone synthase (a key enzyme in the formation of several major classes of flavonoids), thereby achieving a desired characteristic.

One aspect of this invention features a method of producing a transgenic Echinacea plant, such as a transgenic Echinacea purpurea plant. The method includes transforming an Echinacea plant cell, e.g., an Echinacea purpurea cell, with a recombinant nucleic acid, and cultivating the transformed cell to generate a transgenic plant. The recombinant nucleic acid can be introduced into the plant cell by contacting the cell with an Agrobacterium cell, e.g., an Agrobacterium tumefaciens cell, that contains the recombinant nucleic acid. In one embodiment, the plant cell is in a tissue, e.g., leaf, petiole, or root, excised from an Echinacea plant. Preferably, it is excised from a 2-month-old in vitro plantlet. To generate a transgenic plant from the above-mentioned transformed cell, one can culture the cell to generate a somatic embryo, and grow it to generate a whole plant. To select for transformed cells and transgenic plants, the recombinant nucleic acid can be engineered to contain a first sequence encoding a selectable marker, e.g., neomycin phosphotransferase II, so as to provide the transformed cells and transgenic plants a selectable trait, e.g., a kanamycin-resistance. Thus, the above-mentioned somatic embryo can be generated by culturing the transformed cell in a selection medium containing, e.g., kanamycin (e.g., 50 to 100 mg/l) and optionally, timentin to inhibit Agrobacterium cell growth (e.g., 100 to 200 mg/l). The recombinant nucleic acid can also be engineered to contain a second sequence encoding a polypeptide of interest, e.g., a heterologous chalcone synthase. The culturing step is conducted in dark or under dark condition followed by exposure to light.

A nucleic acid refers to a DNA molecule (e.g., a cDNA or genomic DNA), an RNA molecule (e.g., an mRNA), or a DNA or RNA analog. A DNA or RNA analog can be synthesized from nucleotide analogs. The nucleic acid molecule can be single-stranded or double-stranded, but preferably is double-stranded DNA. A “recombinant nucleic acid” is a nucleic acid the structure of which is not identical to that of any naturally occurring nucleic acid or to that of any fragment of a naturally occurring genomic nucleic acid. The term therefore covers, for example, (a) a DNA which has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Specifically excluded from this definition are nucleic acids present in mixtures of different (i) DNA molecules, (ii) transfected cells, or (iii) cell clones, e.g., as these occur in a DNA library such as a cDNA or genomic DNA library. The nucleic acid just described can be used to express a protein in the plant or plant cell. For this purpose, one can operatively link the nucleic acid to suitable regulatory sequences to generate an expression vector.

In another aspect, the invention features a transgenic Echinacea plant, e.g., Echinacea purpurea, whose genome contains a heterologous nucleic acid. The heterologous nucleic acid can encode a heterologous chalcone synthase or a neomycin phosphotransferase II. In a still another aspect, the invention features a transformed Echinacea plant cell, such as a leaf cell, a petiole cell, or a root cell, that contains such a heterologous nucleic acid. A “heterologous” nucleic acid, gene, or protein is one that originates from a foreign species, or, if from the same species, is substantially modified from its original form. For example, a heterologous promoter operably linked to an Echinacea coding nucleic acid sequence is one form of a sequence heterologous to Echinacea. If a promoter and a coding sequence are from the same species, one or both of them are substantially modified from their original forms.

The details of one or more embodiments of the invention are set forth in the accompanying drawings and the description below. Other features, objects, and advantages of the invention will be apparent from the description and from the claims.

### DETAILED DESCRIPTION

This invention is based on a discovery that an Echinacea plant cell can be transformed to express a heterologous polypeptide and used to generate a transgenic plant.

Accordingly, the invention features a method of transforming a recombinant nucleic acid into an Echinacea cell, including that of E. angustifolia, E. pallida, E. paradoxa, and E. purpurea. The transformed cell can be further cultured to generate a transgenic plant.

A recombinant nucleic acid sequence to be introduced contains one or more sequences of interest or fragments thereof. Such sequences are chosen to provide, enhance, suppress, or otherwise modify expression of a desired trait or phenotype in the resulting transgenic plant. Such traits include agronomic traits such as disease resistance, yield, and the like, and quality traits, such as sweetness, flavor, acidity, color, and the like. Exemplary sequences of interest include flavonoid biosynthetic genes, e.g., the chalcone synthase gene, the chalcone isomerase gene, and the dihydroflavonol-4-reductase gene.

The above-mentioned sequence of interest can be a structural gene, which encodes a polypeptide, which imparts the desired phenotype. Alternatively, it may be a regulatory gene, which may play a role in transcriptional and/or translational control to suppress, enhance, or otherwise modify the transcription and/or expression of an endogenous gene within the Echinacea plant. Further, it can be a non-coding sequence, e.g., anti-sense sequence, repress expression of an endogenous gene. A number of constructs can be used in a number of techniques to suppress expression of endogenous plant genes, e.g., sense or antisense suppression or ribozymes. Anti-sense RNA inhibition of gene expression has been described in, e.g., Sheehy et al., Proc. Nat. Acad. Sci. USA, 1988, 85:8805-8809, and Hiatt et al., U.S. Pat. No. 4,801,340. The use of sense DNA sequences to suppress gene expression is described in e.g., Napoli et al. 1990 Plant Cell, 2:279-289 and U.S. Pat. No. 5,283,184.

Structural and regulatory genes of interest may be obtained from depositories, such as the American Type Culture Collection, Rockville, Md. 20852, as well as by isolation from other organisms, typically by the screening of genomic or cDNA libraries using conventional hybridization techniques, such as those described in Maniatis et al., Molecular Cloning—A Laboratory Manual, Cold Spring Harbor Laboratory, Cold Spring Harbor, N.Y. (1989). Screening may be performed by (1) nucleic acid hybridization using homologous genes from other organisms, (2) probes synthetically produced to hybridize to particular sequences coding for desired protein sequences, or (3) DNA sequencing and comparison to known sequences. Sequences for specific genes may be found in various computer databases, including GenBank, National Institutes of Health, as well as the database maintained by the Untied States Patent Office.

To express a heterologous gene in a plant cell, the gene can be combined with transcriptional and translational initiation regulatory sequences, which will direct the transcription of the gene and translation of the encoded protein in the plant cell. For example, for overexpression, a constitutive plant promoter may be employed. A “constitutive” promoter is active under most environmental conditions and states of cell differentiation. Examples of constitutive promoters include the cauliflower mosaic virus (CaMV) 35S transcription initiation region (Odell et al., Nature 313:810-812 (1985)), the 1′- or 2′-promoter derived from T-DNA of Agrobacterium tumafaciens, the ACT11 and Cat3 promoters from Arabidopsis (Huang et al. (1996) Plant Mol. Biol. 33:125-139 and Zhong et al. (1996) Mol. Gen. Genet. 251:196-203), the stearoyl-acyl carrier protein desaturase gene promoter from Brassica napus (Solocombe et al. (1994) Plant Physiol. 104:1167-1176), the GPc1 and Gpc2 promoters from maize (Martinez et al. (1989) J. Mol. Biol. 208:551-565 and Manjunath et al. (1997) Plant Mol. Biol. 33:97-112).

Alternatively, a plant promoter may be employed to direct expression of the gene in a specific cell type (i.e., tissue-specific promoters) or under more precise environmental or developmental control (i.e., inducible promoters). Examples of environmental conditions that may affect transcription by inducible promoters include anaerobic conditions, elevated temperature, the presence of light, spray with chemicals or hormones, or infection of a pathogen. Examples of such promoters include the root-specific ANR1 promoter (Zhang and Forde (1998) Science 279:407) and the photosynthetic organ-specific RBCS promoter (Khoudi et al. (1997) Gene 197:343). For proper polypeptide expression, a polyadenylation region at the 3′-end of the coding region should be included. The polyadenylation region can be derived from the natural gene, from a variety of other plant genes, or from T-DNA.

A recombinant nucleic acid to be introduced preferably carries at least one selectable marker gene to permit screening and selection of transformed cells or somatic embryos derived therefrom (i.e., those cells or embryos which have incorporated the nucleic acid into their chromosomes). The selectable marker gene usually encodes a protein, which permits the survival of transformed cells or somatic embryos in a selective medium. The protein confers the transformants and their progenies antibiotic-resistance (e.g., resistance to bleomycin, Geneticin®, kanamycin, hygromycin, streptomycin, or the like), herbicide resistance (e.g., resistance to chlorosulfuron, a nutritional marker, Basta, or the like), or biocide resistance. The composition of a suitable selective medium is well known in the art. In addition, the recombinant nucleic acid may also contain a reporter gene, which facilitates screening of the transformed cells or somatic embryos for the presence and expression of the exogenous DNA sequence(s). Exemplary reporter proteins include beta-glucuronidase, green-fluorescent-protein, and luciferase.

To generate a transgenic Echinacea plant, the above-described recombinant nucleic acid is introduced to an embryogenic callus, cell suspension, or somatic embryo by incubation with Agrobacterium, e.g., Agrobacterium tumefaciens. The sequence, preferably, is cloned in a transfer DNA (T-DNA) region on a suitable plasmid. Typically, a binary vector system may be used to introduce the sequence. A first plasmid vector would carry the T-DNA sequence while a second plasmid vector would normally carry a virulence (vir) region, which is essential for the transfer of the T-DNA, but is not itself transferred. Transformation can be achieved by incubating Agrobacterium cells carrying both plasmids with the embryogenic callus, cell suspension, or somatic embryo. The virulence functions of the Agrobacterium host will direct the insertion of the recombinant nucleic acid into the plant cell genome when the cell is infected by the bacteria. Agrobacterium-mediated transformation techniques, including disarming and use of binary vectors, are well described in the scientific literature. See, for example, Horsch et al. (1984) Science 233:496-498; Fraley et al. (1983) Proc. Natl. Acad. Sci. USA 80:4803; and Gene Transfer to Plants, Potrykus, ed., Springer-Verlag, Berlin, 1995. The T-DNA is typically modified to delete the tumor inducing one genes present in the T-DNA of wild-type Agrobacterium tumor-inducing (Ti) plasmids. Other suitable plasmids include modified (co-integrate) Ti plasmids. The construction of recombinant binary and Ti plasmids can be accomplished using conventional recombinant DNA techniques, such as those described in Maniatis et al. Frequently, the plasmids include additional selective marker genes, which permit manipulation and construction of the plasmid in suitable hosts, typically E coli. Suitable selective marker genes include those resulting in tetracycline resistance, kanamycin resistance, ampicillin resistance, and the like.

Cells to be transformed can be prepare from two-month-old in vitro explants as described in the example below. Preferably, leaf, petiole, or root cells are contacted with the above-described Agrobacterium cells. After transformation is completed, the Agrobacterium cells were washed away with water or a culture medium. The washing is repeated from two to six times, with suitable antibiotics, e.g., timentin, in at least the later washes in order to kill remaining Agrobacterium cells. After washing, the transformed tissues are placed on a suitable selection medium in dark or under dark condition followed by exposure to light to generate calli and somatic embryos. For example, the cells are cultured under the condition of 16 hour-light/8-hour-dark per day. The medium contains a plant selection agent that permits identification of transformed calli or embryos based on the presence of the marker introduced. Suitable plant selection agents include Geneticin (1-50 mg/l), chlorosulfuron (0.001-0.05 mg/l), and kanamycin (100-500 mg/l). The selection culture is maintained typically for a time sufficient to permit transformed cells to grow and produce calli, somatic embryos, and, finally, shoots, while the non-transformed callus cells and embryos turn brown and die. Typically, the selection culture will last from about 3 to 13 weeks, depending on the concentration and relative activity of the plant selective agent. The primary criterion in ending the selection culture, however, is a clear distinction between proliferating cells, which have been transformed, and non-proliferating cells, which have not been transformed.

The transformed somatic embryos are subsequently transferred to various media for further development into plantlets. The regeneration medium is a general growth medium, such as the one which is described in the example section below, supplemented with a selection agent, and preferably including an anti-Agrobacterium antibiotic. Well developed plantlets can then be transferred to, for example, a greenhouse or elsewhere in a conventional manner for culturing plantlets into whole transgenic plants. A number of tissue culture techniques for in vitro propagation and regeneration from petiole explants of E. purpurea are described in Choffe et al, 2000, In Vitro Cell. Dev. Biol.-Plant 36: 30-36. More recently, axillary buds, adventitious shoots and somatic embryos have been used for in vitro mass propagation of four commercially important Echinacea species, including E. angustifolia, E. pallida, E. paradoxa and E. purpurea (Lakshmanan et al., 2000, J. Horticult. Sci. Biotechnol. 77: 158-163).

Transformation of the resulting plantlets can be confirmed by assaying the plant material for any of the phenotypes that have been introduced by the exogenous DNA. In particular, suitable assays exist for determining the presence of certain reporter genes, such as beta-glucuronidase or luciferase. Other procedures, such as PCR, restriction enzyme digestion, Southern blot hybridization, and Northern blot hybridization may also be used. The presence and copy number of the heterologous sequence in a transgenic plant can be determined using standard methods, e.g., Southern blotting analysis and PCR. Expression of a heterologous gene in a transgenic plant may be confirmed by detecting the mRNA or protein encoded by the gene in the transgenic plant. Means for detecting and quantifying mRNA or proteins are well known in the art.

Once the heterologous sequence has been confirmed to be stably incorporated in the genome of a transgenic plant, it can be introduced into other plants by sexual crossing. Any standard breeding technique can be used, depending upon the species to be crossed.

The above-described method can be used to generate a transformed Echinacea plant cell or a transgenic Echinacea plant that have a desired trait or phenotype. For example, one can express in the plant a heterologous chalcone synthase (e.g., that from Dendranthema grandifora, Gerbera hybrida, Eustoma grandiflorum, Torenia fournieri, or Perilla frutescence). Chalcone synthase (CHS; EC 2.3.1.74) is a key enzyme in the formation of several major classes (flavonols, flavones, isoflavonoids and anthocyanins) of flavonoids. Thus, expression of chalcone synthase may regulate flavonoid levels in cells.

The examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

Plasmid Construction

A full-length 1170 bp cDNA encoding Petunia chalcone synthase (CHS) was PCR-amplified from a violet flower cDNA library of Petunia hybrida cv. Ultra Blue (Koes et al., Nucl. Acids Res. 14 (1986) 5229-5239) and this cDNA was deposited in the GenBank database with an accession number of AF233638. The PCR product was cloned into a pGEM-T Easy vector (Promega) by standard techniques. The resultant clones were digested with EcoRI to release a CHS-encoding fragment. The fragment was then end-filled up by Klenow enzyme, and ligated to the SmaI-SacI-digested (to remove the gus reporter gene) binary vector pBI121 (Chen et al., Mol. Breed. 11, 2003, 287-293). The resulting plasmid, named pCHS, contained a neomycin phosphotransferase (nptII) selection marker gene under the control of a nopaline synthase (nos) promoter and nos terminator. It was 14,065 bp in length and carried a T-DNA segment of 5,500 bp. The Petunia chs cDNA was under the control of the cauliflower mosaic virus (CaMV) 35S promoter and nos terminator. This pCHS vector was then introduced into Echinacea purpurea cells via Agrobacterium-mediated transformation. The T-DNA of pCHS includes the nptII marker gene and facilitates its integration into the genome of a plant cell. Such a plant cell, expressing nptII, could survive in a differentiation medium supplemented with neomycin or kanamycin and further differentiate. In other words, nptII selection marker gene allows one to select for transformants using the antibiotics.

Plant Material and Culture Conditions

Before transformation, effects of kanamycin and timentin on Echinacea purpurea were examined. Timentin, a mixture of ticarcillin and clavulanic acid, is often used for suppressing Agrobacterium tumefaciens growth after transformation (Cheng et al., Plant Cell Rep. 17, 1998:646-649).

Seeds of Echinacea purpurea cv. Magnus were purchased from Royalfleur (Angers, France), sterilized in a 50 ml solution containing 1% sodium hypochloride and 3 drops of Tween 20 for 20 minutes. The seeds were washed thoroughly in sterile deionized water and then germinated on a basal E1 medium containing ½ strength MS salts (Murashige et al., Physiol. Plant. 15 (1962) 473-497) supplemented with 100 mg 1⁻¹ myo-inositol, 2 mg 1⁻¹ glycine, 2% sucrose, and 0.7% phytagar. The pH value of the medium was adjusted to 5.7. The seeds were then incubated in a growth chamber at 25° C. under a cycle of 16-hour illumination (100 μmol m⁻² sec⁻¹) and 8-hour darkness to generate plantlets.

Leaves were obtained from 2-month-old plantlets and cut into small segments. These small segments were inoculated in petri dishes containing an E8 medium, which included MS salts, 100 mg 1⁻¹ myo-inositol, 2 mg 1⁻¹ glycine, 0.5 mg 1⁻¹ BAP, 0.5 mg 1⁻¹ NAA, 2% sucrose, and 0.7% phytagar supplemented with various concentrations of kanamycin (“k”) or/and timentin (“t”) (Duchefa, The Netherlands). More specifically, eight groups of leaf explants (30/group) were isolated. One group was cultured in E8 media free of kanamycin or timentin (“E8”). Four groups were cultured in E8 media supplemented with 10 mg 1⁻¹, 20 mg 1⁻¹, 40 mg 1⁻¹, and 70 mg 1⁻¹ kanamycin (“E8/k10,” “E8/k20,” “E8/k40,” and “E8/k70”). Two groups were cultured in E8 media supplemented, respectively, with 100 mg 1⁻¹ and 200 mg 1⁻¹ timentin (“E8t/100” and “E8/t200”). One group was cultured in presence of 100 mg 1⁻¹ kanamycin and 200 mg 1⁻¹ timentin (“E8/k100/t200”). The explants in each group were grown in three Petri dishes (10 leaf explants/dish) and were kept in a growth chamber at 25° C. under a cycle of 16-hour illumination (100 μmol m⁻² sec⁻¹) and 8-hour darkness to generate plantlets for up to 3 months. Effects of kanamycin and timentin are summarized in Table 1 below:

TABLE 1
Effects of kanamycin or timentin

| | No. of explants | No. of explants | No. of explants | No. of explants | No. of explants |
|---|---|---|---|---|---|
| | without | producing | producing | producing | turning to |
| Medium | differentiation | callus | somatic embryo | shoot | death |
| E8 | 0 | 30 | 30 | 28 | 0 |
| E8/k10 | 18 | 12 | 0 | 0 | 0 |
| E8/k20 | 30 | 0 | 0 | 0 | 0 |
| E8/k40 | 20 | 0 | 0 | 0 | 10 |
| E8/k70 | 11 | 0 | 0 | 0 | 19 |
| E8/t100 | 0 | 30 | 30 | 0 | 0 |
| E8/t200 | 0 | 30 | 30 | 28 | 0 |
| E8/k100/t200 | 4 | 0 | 0 | 0 | 26 |

Note:
At the end of the culture period, some explants had no morphological changes (e.g., no callus induction, green leaf blades) and were scored as “without differentiation.”

It was estimated that the minimal concentration of kanamycin for selection was 50 mg 1⁻¹. Further, timentin showed no toxicity to Echinacea tissue even at a concentration of 200 mg 1⁻¹. Interestingly, timentin at 200 mg 1⁻¹ stimulated shoot induction in leaf explants as compared to that at 100 mg 1⁻¹. It is known that timentin promotes shoot regeneration of tobacco (Nauerby et al., Plant Sci. 1997, 123:169-177) at 150 mg 1⁻¹ and increases the morphogenesis of tomato cotyledon explants at 300 mg 1⁻¹ (Costa et al., Plant Cell Rep. 2000, 19: 327-332). In all experiments described below, 50 mg 1⁻¹ kanamycin and 200 mg 1⁻¹ timentin were used.

The above-described pCHS vector was electroporated into the Agrobacterium tumefaciens strain LBA4404 by electroporation (Bio-Rad Gene Pulser II, Bio-Rad Laboratories, Inc., Hercules, Calif.) using standard techniques. Transformed Agrobacterium bacteria were was then used for plant transformation. More specifically, the bacteria were inoculated in 50 ml AB medium (5 g 1⁻¹ glucose; 1 g 1⁻¹ NH₄Cl; 0.3 g 1⁻¹ MgSO₄.7H₂O; 0.15 g 1⁻¹ KCl; 10 mg 1⁻¹ CaCl₂; 2.5 mg 1⁻¹ FeSO₄.7H₂O; 3 g 1⁻¹ K₂HPO₄; 1.15 g 1⁻¹ NaH₂PO₄.H₂O) supplemented with 100 μg ml 1⁻¹ kanamycin and incubated for 1 day at 28° C. Acetosyringone (AC; Fluka Chemie AG, Buchs, Sankt Gallen, Switzerland) and glucose were added to the AB/kanamycin medium to yield a final concentration of 100 μM and 5%, respectively. After the culture was incubated for 4 hours at 28° C., the Agrobacterium were centrifuged at 5,000 rpm for 5 minutes. The pellet was resuspended in a KCMS medium (MS salts; 0.9 mg 1⁻¹ thiamine; 0.2 mg 1⁻¹ 2,4-D; 200 mg 1⁻¹ potassium acid; pH 5.6) supplemented with 100 μM AS and 5% glucose. The resuspension was then diluted such that the final OD₆₀₀ reading was 0.8 to 1.2, and was used to transform Echinacea purpurea cells.

Different tissues of Echinacea. purpurea were examined to determine their competency for regeneration and transformation. Again, leave, petiole, and root explants were excised from in vitro grown E. purpurea plantlets, cut into small segments, and immersed in the above-described Agrobacterium suspension for 1 hour. Excess suspension was blotted from the explants with filter paper. Then, the infected explants were transferred onto a KCMS solid medium supplemented with 100 μM AC and 5% glucose and left to grow for 5 days in a dark growth chamber at 25° C. Afterwards, the explants were washed in a solution containing 200 mg 1⁻¹ timentin, transferred to an E8 medium supplemented with 200 mg 1⁻¹ timentin and 50 mg 1⁻¹ kanamycin, and incubated at 25° C. in a growth chamber with a photoperiod of 16-hour light (60 μmol m⁻² sec⁻¹) and 8-hour dark or totally in the dark. Approximately 3 to 4 weeks later, surviving calli were observed at the edges of the tissue explants. Within 3 months, somatic embryos appeared. Some of them contained were shoot buds, and were excised and transferred into a shooting medium (a basal E1 medium supplemented with 0.1 mg 1⁻¹ BAP and 50 mg 1⁻¹ kanamycin). The culture was incubated in a 25° C. growth chamber with a 16-hour photoperiod/day. The regenerated shoots were then transferred to a fresh basal E1 medium in the same growth chamber. The rooted plantlets were then transferred to potting soil mixtures of finnpeat (Kekkila, Finland) and King Root Gardening number 3 (King Root Gardening Co., Taiwan) in green house. The results are summarized in Table 2 below.

TABLE 2
Competency of different E. purpurea
tissues for differentiation/transformation

| Tissue ex- | No. of explants | No. of explants | No. of explants | |
|---|---|---|---|---|
| plants (no. | producing | producing | producing shoot | |
| of explants) | callus | somatic embryos | (% of all explants) | |
| Leaf (19) | 15 | 14 | 9 | (47%) |
| Petiole (61) | 32 | 5 | 1 | (2%) |
| Root (65) | 17 | 5 | 3 | (5%) |

As shown in Table 2, 47% of total leaf explants generated calli and further produced shoots, whereas the percentage of regenerated shoots from petiole and root tissues were less than 5%.

The effect of light on shoot regeneration was also investigated. More specifically, leaf explants were transformed with Agrobacterium in the manner described above. Then, the explants were transferred onto a KCMS solid medium and co-cultured with Agrobacterium for 5 days in a 25° C. growth chamber under a cycle of 16-hour light/8-hour dark (“Light”) or completely in the dark (“‘Dark”’). The explants were then transferred onto a selection E8 medium supplemented with kanamycin (50 mg 1⁻¹) and timentin (200 mg 1⁻¹) and cultured for up to 3 months in a 25° C. growth chamber under a cycle of 16-hour light/8-hour dark (“Light”) or completely in the dark (“Dark”). It was found that within 3 months, the explants not transformed turned yellow, and produced no calli. On the contrary, those transformed survived and produced callus. The results are summarized in Table 3 below.

TABLE 3
Effects of illumination on differentiation of Echinacea purpurea
leaf explants transformed by Agrobacterium tumefaciens

| | Culture | No. of explants | No. of explants | No. of explants | |
|---|---|---|---|---|---|
| Co-culture | conditions (no. | producing | producing | producing shoot | |
| conditions | of leaf discs) | callus | somatic embryo | (% of total explants) | |
| Light | Light (27) | 4 | 0 | 0 | (0%) |
| | Dark (36) | 16 | 2 | 2 | (6%) |
| Dark | Light (87) | 67 | 24 | 15 | (18%) |
| | Dark (21) | 17 | 6 | 6 | (29%) |

As shown in Table 3, a higher percentage (29%) shoot regeneration was obtained from leaf explants co-cultured with Agrobacterium in the dark and then cultured in the selection medium in the dark.

Under the above described conditions, 11 plants were generated. They were further analyzed by genomic PCR and Southern blot. Total genomic DNA was extracted from green leaves of the 11 transformants and wild-type plants using the CTAB method (Wilkie, Isolation of total genomic DNA, in: M. S. Clark (ed), Plant Molecular Biology—A Laboratory Manual, Springer-Verlag Berlin, 1997, pp. 3-15). PCR was carried out with the following primer sets: Kan-F (5′-ATGATTGAACAAGATGGA-3′) and Kan-R (5′-TCAGAAGAACTCGTCAAG-3′) for amplifying a 795 bp full-length nptII gene sequence (Chen et al., Mol. Breed. 2003, 11:287-293); and CHS-F1 (5′-ATGGTGACAGTCGAGGAG-3′) and CHS-R1 (5′-TTAAGTAGCAACACTGTG-3′) for amplifying a 1170 bp Petunia chs cDNA full-length sequences (GenBank accession number AF233638). The PCR mixture (20 μl) was denatured for 5 minutes at 94° C. prior to 30 amplification cycles (1 minute at 94° C., 1 minute at 55° C., 1 minute at 72° C.). Finally an extension reaction was performed at 72° C. for 10 minutes. Afterwards, 5 μl of the PCR product was run on 1% agarose gel to check the fidelity of the gene product. It was found that no PCR product was produced from wild type plant. In contrast, the 795-bp and 1170-bp fragments were detected in PCR products from five plants. These five plants were confirmed to be transgenic plants.

Southern blot analysis was carried out to estimate transgene copy number. More specifically, genomic DNA from green leaves of each plant was isolated according to the method described in To et al., Planta, 1999, 209:66-76). Twenty microgram of purified DNA was digested overnight at 37° C. with EcoRI or HindIII. After electrophoresis on a 0.8% agarose gel in TAE, the DNA was transferred onto an Hybond-N+ nylon membrane (Amersham Pharmacia Biotechnol.). To prepare a non-radioactive PCR DIG hybridization probe, equal amounts (20 pmol) of the forward primer 35S-Pro-F1 (5′-AGATTAGCCTTTTCAATT-3′) and the reverse primer CHS-R1 (5′-TTAAGTAGCAACACTGTG-3′) were used to amplify a chimeric fragment (2033 bp) containing the CaMV 35S promoter and Petunia chs cDNA from the expression vector pCHS (100 ng) in a reaction mixture (5 μl of 10× Taq buffer, 1 μl each of 10 mM dATP, 10 mM dGTP, 10 mM dCTP, 0.9 μl of 10 mM dTTP, 1 μl of 1 mM dig-11-dUTP, and 0.2 μl of Taq DNA polymerase). After the total volume of the mixture was adjusted to 50 μl, the PCR was carried out for 30 cycles (1 minute at 94° C., 1 minute at 55° C., 1 minute at 72° C.). A final extension was performed at 72° C. for 10 minutes. Following purification, 1 μl of PCR product was run on a 1% agarose gel to check the accuracy of the PCR probe. 10 μl of PCR product was used to hybridize the membrane overnight at 65° C. in 10 ml of a hybridization solution (5× SSPE; 0.5% laurylsarcosine; 1% SDS; 1% blocking reagent). The membrane was washed twice in a washing solution (0.2% SSPE; 0.1% SDS) at 65° C. for 10 minutes and once in buffer 1 (0.1 M maleic acid, pH 7.5; 0.15 M NaCl; 0.3% Tween 20) at room temperature for 20 minutes, and then incubated at room temperature for 30 minutes in 10 ml of buffer 2 (0.1 M maleic acid, pH 7.5; 0.15 M NaCl; 0.5% blocking reagent). It was incubated in 5 ml of anti-dig-AP solution (anti-dig-AP 1:10,000 diluted in buffer 2) at room temperature for 30 minutes. After washing twice with buffer 1 for 10 minutes and once with buffer 3 (0.1 M Tris, pH 9.5; 0.1 M NaCl; 0.05 M MgCl₂) for 2 minutes, the membrane was incubated with 0.5 ml of a 1× CSPD solution in a bag. The membrane was incubated at 37° C. for 10 minutes, exposed to X-ray film for 1.5 hours, and then developed for 1 to 5 minutes.

It was found that no band was detected in wild-type DNA. One band was detected in transformant Ep4, suggesting that 1 copy of the transgenic cassette was integrated into the plant genome. Two hybridization bands were detected in transformant Ep5, suggesting 2 copies of transgenic cassette in the plant genome. Multiple hybridization bands were detected in Ep1, Ep6, and Ep11, suggesting multiple copies in these transgenic plants' genomes.

To further confirm the genomic integration of T-DNA from the pCHS vector, HindIII-digested plant genomic DNA was probed with a 0.8 kb DNA fragment containing the bacterial NPTII coding region. As expected, no hybridization band was detected in wild-type Echinacea plants. In contrast, one and two bands were observed in transgenic plants Ep4 and Ep5, respectively. Multiple bands were observed in plants Ep1, Ep6, and Ep11. These results were consistent with those described above.

The 5 transgenic plants were grown to maturity. It was found that the transgenic plants produced red flowers. Also, white sectors were observed on mature petals of the plants. In contrast, wild type plants produced pale red flowers with no white sector.

To examine tissue expression of the transgene, total RNA was isolated from different tissues and probed with the Petunia chs probe. No chs mRNA signal was detected in RNA from all tissues examined, including root, leaf, and flower in wild-type plants and plants Ep1 and Ep1. A strong signal and a much weaker signal were respectively detected in RNAs isolated from petals and leaves of transgenic plant Ep5. No chs mRNA was observed in its root. For transgenic plant Ep4, a very weak signal and a relatively strong signal was detected in petals and roots, respectively; and none was detected in leaves. Transgenic plant Ep6 was still at the vegetative stage under green house conditions. However, a relatively higher expression of chs mRNA was found in its leaves and a weak signal detected in its roots.

The Agrobacterium-mediated transformation has been used for introducing new genes into plants, and for inactivating genes by T-DNA insertion mutagenesis (Tinland, Trends Plant Sci. 1996, 1:178-184). To characterize the plant/T-DNA insertion sites in the above-described transgenic plants, an inverse PCR (IPCR) (Chen et al., Mol. Breed. 2003, 11:287-293) was used to determine the T-DNA insertion sequences in two selected transgenic plants, Ep4 and Ep5, which carried one and two copies of the transgene, respectively.

Unique plant sequence was identified near the right border of T-DNA in plants Ep4 and Ep5. Only a single insertion was detected in the genome of plant Ep4, which is consistent with the result of the above-described Southern blot. A unique 366 bp plant sequence (shown below) in the Ep4 plant had no similarity to sequences in public nucleotide and expressed sequencing tag (EST) databases.

TCGAGGTCATCTCTTCATTACCACAAACGTACTACAACTCGTTCGTCTTG
TCTTCCACAAACGATTAAACATATAGAAAAACTTGATGTTGTTTCAGACT
CTCATCTCCGTTAGCACAACTACGGTTGCTTTACCTTGGCAGACATATAC
GCAACCTAAAGAAAAATATAAAAATTTATTTTAAGAGATCCGTGTTAAAG
AGATTCATGATCAACATCTGACGTCTCTTACATGCTCATTCTTGAAGGTG
TAAACCGTATTATGTTTTATTGTTATCCAAACAAAACCTCTAAGGATAAG
GTTTAGTTTGTTTTCATAATGGTCATGGTCAATAACGTGTTAGAAACGTG
AAAAGTGCATCTCATA

For plant Ep5, two PCR bands were obtained when suing the forward primer IP2 (5′-TTGTCAAGACCGACCTGTCCG-3′) and the reverse primer IP1 (5′-CGTTGCGGTTCTGTCAGTTCC-3′). However, only a single band (ca. 0.7 kb) was found when another nested primer set (forward primer IPCR-P2-1 and reverse primer IPCR-P1-1) were employed for second-round PCR amplification. After cloning and sequencing, a 528 bp sequence (shown below) was obtained. Database searching indicated that it had no sequence similarity to other known nucleotide sequences.

TCGACTACTTACAACCTATTGAATGATAGTTGTTTCTAGCACAAGAAATT
GTTCTCTGACTTGTAAAATTTAGACAACTTTTGTTATACAAATTAGTCAA
ATTGTTAACAAAGATAAGCTACTTAAAGCATCTAAAATGTACAAAAAGTT
GTAATGTGTATTAGACAAACACATACAAAGGATAAAAGTCCATCCATAAG
AAATATGATAAATGTTAAAAGACAATCTGAAGTTATAAATTCAGTCACAT
GTATCTTTCTATCTCCCCCTTGTCACCGTTATTAATGTTATTCTCGGATT
CATCAGTCATCCCTTGACATATTTAATCCTAGCTTAACTCTTAATCACGA
TGTGAAACTATAAATTCAGTCACATGTATGATGCTGACATATCAAATTTA
TATATTGCTTCGTGGCAAATGTTCATTAAATCTGTAATAAAGATCGACTA
GATTTCGTCCCAACTTCAGCCAGTTTTCTACAATGTGTCAACAGACCATG
TCTACTTTTTTCCCAGCCGTTCCTCACC

Further, the right border region of the vector pCHS contained a 162-bp region including a 25-bp direct repeat and flanking sequences. In contrast, the genome of Ep4 or Ep5, a 42-nucleotide region and a 36-nucleotide region were retained, respectively, suggesting that these two plants were independently transgenic lines.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A method of producing a transgenic Echinacea plant, the method comprising:
transforming an Echinacea plant cell with a recombinant nucleic acid, and cultivating the transformed cell to generate a transgenic Echinacea plant.

2. The method of claim 1, wherein the Echinacea plant is Echinacea purpurea.

3. The method of claim 2, wherein the recombinant nucleic acid is introduced into the Echinacea plant cell by contacting the cell with an Agrobacterium that contains the recombinant nucleic acid.

4. The method of claim 3, wherein the Agrobacterium is Agrobacterium tumefaciens.

5. The method of claim 4, wherein the cell is in a plant tissue excised from an Echinacea plant.

6. The method of claim 5, wherein the plant tissue is leaf, petiole, or root.

7. The method of claim 5, wherein the plant tissue is excised from a 2-month-old in vitro plantlet.

8. The method of claim 5, wherein the cultivating step is conducted by
culturing the transformed cell to generate a somatic embryo, and
growing the somatic embryo to generate the plant.

9. The method of claim 8, wherein the recombinant nucleic acid contains a first sequence encoding a neomycin phosphotransferase II or a second sequence encoding a heterologous chalcone synthase.

10. The method of claim 10, wherein the somatic embryo is generated by culturing the transformed cell in a selection medium containing kanamycin or timentin.

11. The method of claim 10, wherein the kanamycin is 50 to 100 mg/l and the timentin is 100 to 200 mg/l.

12. The method of claim 5, wherein the culturing step is conducted in dark or under dark condition followed by exposure to light.

13. The method of claim 1, wherein the recombinant nucleic acid is introduced into the Echinacea plant cell by contacting the cell with an Agrobacterium that contains the recombinant nucleic acid.

14. The method of claim 13, wherein the Agrobacterium is Agrobacterium tumefaciens.

15. The method of claim 14, wherein the cell is in a plant tissue excised from an Echinacea plant.

16. The method of claim 15, wherein the plant tissue is leaf, petiole, or root.

17. The method of claim 15, wherein the plant tissue is excised from a 2-month-old in vitro plantlet.

18. The method of claim 15, wherein the cultivating step is conducted by
culturing the transformed cell to generate a somatic embryo, and
growing the somatic embryo to generate the plant.

19. The method of claim 15, wherein the culturing step is conducted in dark or under dark condition followed by exposure to light.

20. The method of claim 1, wherein the cell is in a plant tissue excised from an Echinacea plant.

21. The method of claim 20, wherein the plant tissue is leaf, petiole, or root.

22. The method of claim 20, wherein the plant tissue is excised from a 2-month-old in vitro plantlet.

23. The method of claim 20, wherein the cultivating step is conducted by
culturing the transformed cell to generate a somatic embryo, and
growing the somatic embryo to generate the plant.

24. The method of claim 5, wherein the culturing step is conducted in dark or under dark condition followed by exposure to light.

25. A transgenic Echinacea plant whose genome comprises a heterologous nucleic acid.

26. The plant of claim 25, wherein the Echinacea plant is Echinacea purpurea.

27. The plant of claim 26, wherein the heterologous nucleic acid encodes a heterologous chalcone synthase or a neomycin phosphotransferase II.

28. A transformed Echinacea plant cell comprising a heterologous nucleic acid.

29. The cell of claim 28, wherein the Echinacea plant is Echinacea purpurea.

30. The cell of claim 29, wherein the heterologous nucleic acid encodes a heterologous chalcone synthase or a neomycin phosphotransferase II.

31. The cell of claim 28, wherein the cell is a leaf cell, a petiole cell, or a root cell.

32. The cell of claim 31, wherein the heterologous nucleic acid encodes a heterologous chalcone synthase or a neomycin phosphotransferase II.
